(19) United States
(12) Patent Application Publication
(10) Pub. No.: US 2026/0240868 A1
(43) Pub. Date: Aug. 20, 2026

(54) USE OF CARBENOXOLONE FOR TREATMENT OF DEMYELINATING NEUROLOGICAL DISORDERS

(71) Applicant: KOC UNIVERSITESI, Istanbul (TR)

(72) Inventors: Yasemin Gursoy Ozdemir, Istanbul (TR); Ege Anil Ucar, Istanbul (TR); Esra Ozkan, Istanbul (TR); Narges Shomalizadeh, Istanbul (TR); Emine Sekerdag Kilic, Istanbul (TR); Fatmanur Akpunar Salman, Istanbul (TR); Selin Sapanci, Istanbul (TR); Judi Kesebi, Istanbul (TR); Ceyda Ozler, Istanbul (TR); Alara Su Bilgez, Istanbul (TR)

(73) Assignee: KOC UNIVERSITESI, Istanbul (TR)

(21) Appl. No.: 19/192,432

(22) Filed: Apr. 29, 2025

(30) Foreign Application Priority Data
Feb. 17, 2025 (TR) 2025/001828

(51) Int. Cl. A61K 31/56; A61K 45/06; A61P 25/28

(52) U.S. Cl. CPC A61K 31/56; A61K 45/06; A61P 25/28

(57) ABSTRACT

A use of Carbenoxolone (CBX), a hemichannel blocker, as a therapeutic agent for treating demyelinating neurological disorders, particularly Multiple Sclerosis (MS), is provided. By inhibiting hemichannels in pericytes, Carbenoxolone reduces fibrosis around blood vessels, preserving blood-brain barrier integrity, minimizing lesion formation, and ultimately slowing neurodegeneration. This MS management is provided, focusing on neurovascular protection and antifibrotic action to address both relapsing-remitting and progressive forms of the disease.

### CROSS REFERENCE TO THE RELATED APPLICATIONS

This application is based upon and claims priority to Turkish Patent Application No. 2025/001828, filed on Feb. 17, 2025, the entire contents of which are incorporated herein by reference.

### TECHNICAL FIELD

The invention relates to the use of Carbenoxolone, a hemichannel inhibitor, for the therapeutic treatment of demyelinating neurological disorders, specifically focusing on interventions for conditions like Multiple Sclerosis (MS).

### BACKGROUND

Multiple Sclerosis (MS) is a chronic autoimmune, demyelinating neurological disorder that primarily affects the central nervous system (CNS) and remains a leading cause of non-traumatic disability in young adults. The prevalence of MS has been steadily increasing worldwide, particularly in developed countries. MS can present in different forms, with the majority of patients (approximately 85%) experiencing the relapsing-remitting type (RRMS), while others develop progressive forms such as primary-progressive MS or late-onset MS. As the disease progresses, patients often suffer from cognitive dysfunction and progressive neurological deterioration, contributing significantly to long-term disability and presenting a heavy burden on both individuals and society. A significant contributor to this progression is neurodegeneration and brain volume loss, which continue to occur independently of the inflammatory processes traditionally associated with MS [1].

Current Disease-Modifying Therapies (DMTs) have been developed to modulate immune activity and reduce the frequency and severity of MS relapses. These include interferons, glatiramer acetate, and monoclonal antibodies like natalizumab, alongside sphingosine-1-phosphate (S1P) receptor modulators such as fingolimod. Although these treatments have been effective in reducing new lesion formation and managing inflammation, they do not fully address the neurodegenerative processes that underlie progressive MS, particularly the non-relapsing, slowly expanding lesions often associated with smoldering or chronic active disease. These lesions are characterized by persistent neurodegeneration, which is not adequately targeted by current therapies focused primarily on immune modulation. In addition to their limitations in preventing progressive disability, DMTs can impose significant financial and personal burdens. The cost of these therapies is substantial, and patients undergoing long-term treatment often face a high economic burden, as well as potential side effects associated with prolonged immune suppression [2].

Recent research has highlighted the role of fibrosis in MS pathophysiology, particularly perivascular fibrosis, which contributes to long-term neurodegeneration. Fibrosis occurs when pericytes, which are essential components of the neurovascular unit responsible for maintaining the blood-brain barrier (BBB), respond to BBB breakdown. These cells migrate to the site of injury and differentiate into myofibroblasts, depositing extracellular matrix components such as collagen and fibronectin, which leads to thickening of the vessel walls and fibrosis. This process exacerbates neurodegenerative damage, contributing to disease progression in MS. Studies using the proteolipid protein (PLP)-induced experimental autoimmune encephalomyelitis (EAE) model, a common animal model of RRMS, have demonstrated that perivascular fibrosis plays a significant role in disease progression by promoting scar formation and thickening of the vessel wall, especially in areas of BBB dysfunction. These findings emphasize the importance of targeting pericyte-mediated fibrosis in order to halt or reverse the progression of MS [3].

Pericytes have been studied extensively in other models of fibrosis, such as in the liver and lungs, where their role in scar tissue formation and fibrosis has been well-documented. Their role in MS, however, is only recently being understood. Pericytes are known to be one of the first responders to systemic neuroinflammation, and in MS, they attempt to maintain the integrity of the BBB by increasing their coverage of the endothelial wall. However, in doing so, they also contribute to the formation of fibrotic lesions, especially in progressive MS, where inflammation is less pronounced, and neurodegeneration predominates. Studies have shown that in MS, pericytes accumulate at sites of BBB breakdown, leading to perivascular fibrosis and the formation of chronic active lesions, often referred to as smoldering lesions. These lesions, characterized by slowly expanding neurodegenerative damage, are a key driver of long-term disability in progressive MS [4].

In the prior art, U.S. Pat. No. 8,399,514B2 relates to a therapeutic approach for treating neurological disorders, including Multiple Sclerosis (MS), through the use of sphingosine-1-phosphate (S1P) receptor modulators. These compounds work by inhibiting lymphocytes from exiting lymph nodes, thus reducing immune cell infiltration into the central nervous system and subsequently lowering inflammation and the formation of new lesions. This immune-modulating approach aims to manage MS symptoms, especially by reducing relapses associated with inflammatory responses. However, its inability to address progressive neurodegeneration and non-inflammatory mechanisms prevalent in advanced MS stages, as well as blood-brain barrier dysfunction and fibrotic changes around lesions, contribute significantly to long-term disability. Additionally, the immune-suppressing nature of SIP receptor modulators poses risks of side effects such as increased infection susceptibility, and the need for ongoing treatment creates a substantial financial burden on patients.

These limitations specifically, the inability of SIP receptor modulators to address progressive neurodegeneration in advanced MS, their lack of efficacy in managing non-inflammatory mechanisms like smoldering lesions, unaddressed blood-brain barrier dysfunction and fibrotic changes around lesions, increased risks of side effects from prolonged immune suppression, and the high financial burden of continuous treatment, highlight the need for new therapeutic strategies that go beyond immune modulation to address the complex, progressive neurodegenerative aspects of MS, providing more effective long-term management of the disease and its disability impacts.

### SUMMARY

The invention provides a therapeutic approach for treating demyelinating neurological disorders, particularly Multiple Sclerosis (MS), by targeting fibrotic mechanisms within the central nervous system. Unlike traditional treatments that primarily focus on immune modulation, this invention addresses the underlying fibrosis and neurovascular dysfunction contributing to disease progression and long-term disability in MS.

The invention aims to reduce perivascular fibrosis, thereby preserving blood-brain barrier integrity and preventing lesion formation associated with MS progression.

Another aim of the invention is to mitigate chronic neurodegeneration by addressing the fibrotic changes around MS lesions, which are not targeted by existing immune-modulating therapies.

The invention also aims to provide a therapeutic approach by demonstrating that the presence of pannexin 1 (panx1) in HBVP cells allows for the antifibrotic properties of carbenoxolone, a pannexin 1 haemichannel blockerThe invention also aims to provide a therapeutic approach that minimizes the risk of side effects from prolonged immune suppression, offering a safer alternative for long-term MS management.

Additionally, the invention seeks to provide a cost-effective therapeutic solution by reducing the need for continuous immune-suppressing treatments and their associated economic burdens on patients and healthcare systems.

### BRIEF DESCRIPTION OF THE DRAWINGS

FIGS. 1A-1K show in vitro cellular response of cultured BBB components against human MS serum.

FIG. 2 shows increased pericyte proliferation upon exposure to MS serum.

FIGS. 3A-3H show analysis of PLP EAE induced fibrotic areas in spinal cord and cerebellum sections.

FIGS. 4A-4C show increased fibrotic parameters in EAE.

FIGS. 5A-5H show PDGFRβ+ perivascular cells tightly cover leaking vessels located in fibrotic areas in PLP EAE spinal cord sections.

FIGS. 6A-6D show the validation of PLP-EAE model; improvement of clinical scores and decrease in demyelinated areas upon treatment with carbenoxolone.

FIGS. 7A-7B show reversal of the fibrosis upon treatment with Intracerebroventricular carbenexolone treatment in EAE model.

FIGS. 8A-8B show the collagen 3 expressions in pericyte culture treated with MS serum and reversal of fibrosis on pericytes upon exposure to carbenoxolone.

FIG. 9 shows the fibronectin expressions in pericyte culture treated with MS serum and decrease of fibronectin related fibrosis with treatment of carbenoxolone.

FIG. 10 shows the presence of pannexin 1 (Panx1) channels on human brain vascular pericytes (HBVP).

### DETAILED DESCRIPTION OF THE EMBODIMENTS

The invention relates to the therapeutic use of Carbenoxolone (Formula I) wherein ingredient is carbenexolone disodium powder and chemical name of the molecule: 3βdroxy-11-oxoolean-12-en-30-oic acid 3-hemisuccinate for treating demyelinating neurological disorders, specifically Multiple Sclerosis (MS), by targeting perivascular fibrosis within the central nervous system (CNS), which is shown below:

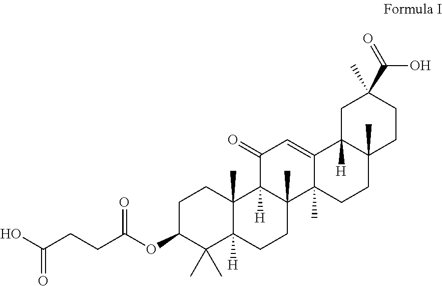

Carbenoxolone acts as an antifibrotic agent by inhibiting hemichannels in pericytes, which are located around blood vessels and play a crucial role in maintaining blood-brain barrier (BBB) integrity. In MS, pericytes contribute to fibrosis when they differentiate into myofibroblasts, leading to the deposition of extracellular matrix components that thicken the vessel walls. This fibrotic buildup disrupts the BBB, promotes lesion formation, and accelerates neurodegeneration. Carbenoxolone prevents this process by blocking hemichannels, thereby reducing fibrosis, preserving BBB integrity, and inhibiting lesion expansion, addressing the progressive neurodegenerative features of MS.

In experimental study, 8-12-week-old female BalbC mice were used to investigate the effects of Carbenoxolone (CBX) on Multiple Sclerosis (MS) using the proteolipid protein (PLP)-induced experimental autoimmune encephalomyelitis (EAE) model. The mice were divided into four groups: Sham, proteolipid protein-induced experimental autoimmune encephalomyelitis (PLP-EAE), CBX intraperitoneal (IP), and CBX intracerebroventricular (ICV). The Sham group only received an adjuvant, while the PLP-EAE group was immunized with PLP139-151 antigen and pertussis toxin to induce the MS model. The CBX-treated groups received CBX either intraperitoneally or intracerebroventricularly. Neurological damage and disease progression were monitored using clinical scoring (0-5 scale) and body weight measurements every two days for 20 days. Clinical evaluations showed that both CBX-treated groups exhibited significant improvements compared to the untreated PLP-EAE group. Neurological deficits in the PLP-EAE group worsened progressively, while the CBX-treated groups showed reduced symptoms and earlier remission. After the 20-day experiment, brain and spinal cord samples were collected, and immunofluorescence staining was performed for myelin-basic protein (MBP), collagen, and fibronectin, to assess demyelination and fibrosis. The results showed significant improvements in the CBX-treated groups, with reduced demyelination and fibrosis, particularly in the CBX ICV-treated group. In vitro experiments using human brain vascular pericytes (HBVPs) cultured with MS patient serum also showed that CBX significantly reduced collagen and fibronectin production, demonstrating its antifibrotic effects. Experimental studies, including the proteolipid protein (PLP)-induced experimental autoimmune encephalomyelitis (EAE) model, demonstrated Carbenoxolone's efficacy. In this model, fibrosis appeared predominantly in perivascular areas, with PDGFRβ-positive pericytes accumulating around affected vessels and contributing to extracellular matrix deposition. Carbenoxolone was shown to significantly reduce pericyte-driven fibrosis and improve clinical outcomes by reducing demyelination and decreasing lesion size. In human brain vascular pericyte cultures exposed to MS patient serum, Carbenoxolone effectively reduced fibrotic extracellular matrix deposition, further supporting its neuroprotective role.

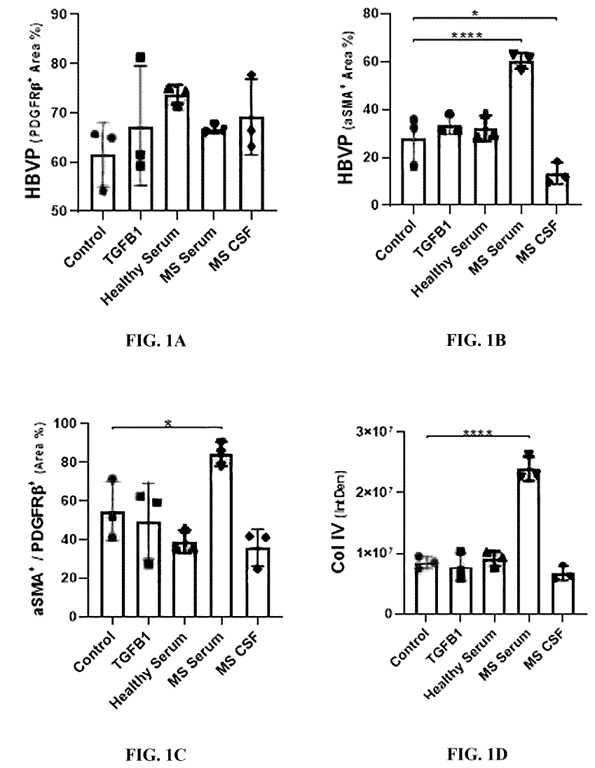

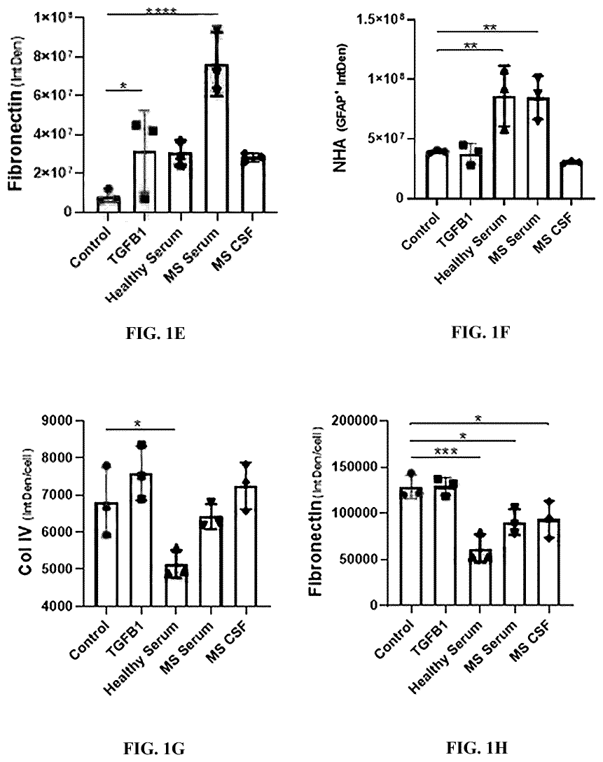

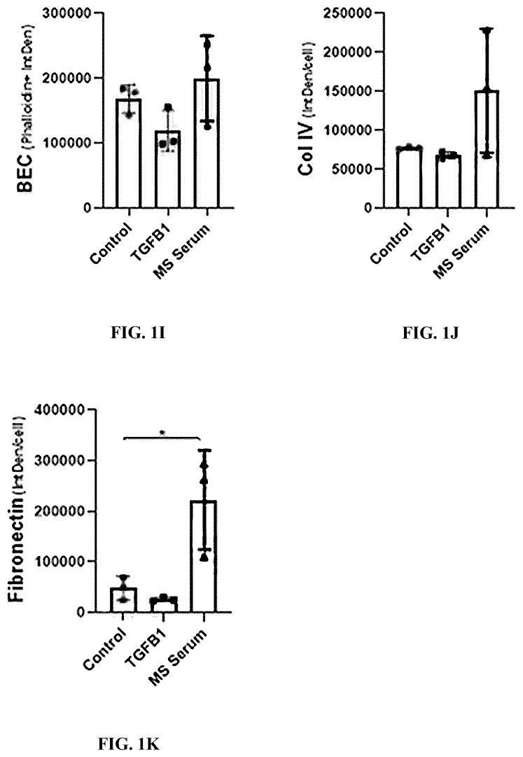

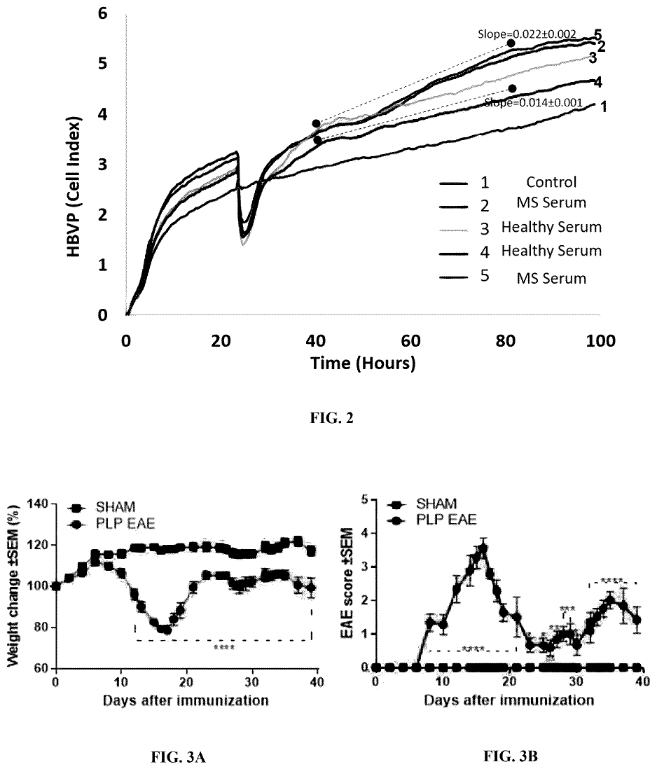

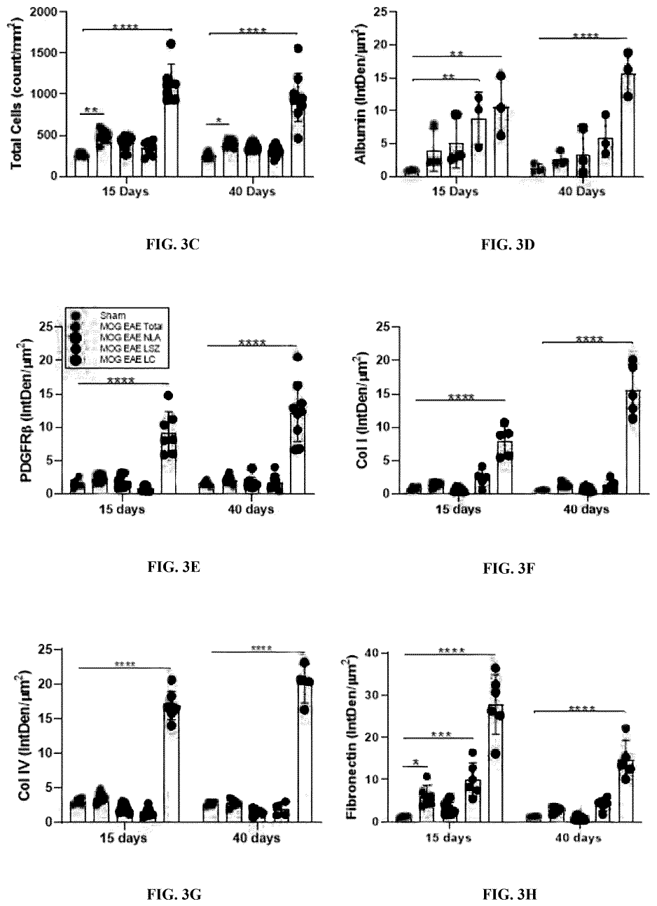

FIGS. 1A-1K show in vitro cellular response of cultured BBB components against human MS serum. Human brain vascular pericytes (HBVP) were exposed to either pericyte medium only (control), TGFB1, healthy human serum, human MS serum, or CSF for 72 hours. Expression analysis of Immunofluorescence-stained images of HBVP (FIGS. 1A-1E), NHA (FIGS. 1F-1H), and BEC (FIGS. 1I-1J) are shown. Cultured HBVP were stained for PDGFRB (pericyte marker) and aSMA (smooth muscle cell/myofibroblast marker), Collagen IV (ECM), Fibronectin (ECM) and phalloidin (cytoskeletal structure). Cells were counterstained with DAPI for cell nuclei (blue). Cultured NHA were exposed to either astrocyte medium only (control), TGFB1, healthy human serum, human MS serum, or CSF for 72 hours and stained for GFAP (astrocyte marker), Collagen IV, or Fibronectin. BEC were exposed to only endothelial medium (control), TGFB1, or human MS serum and were stained for Collagen IV and Fibronectin. Cells were counterstained with DAPI for cell nuclei (blue) and phalloidin for cytoskeletal cell structure. All graphical data are shown as scatter plot diagrams with mean±SD for n=3 independent cell cultures with sera and CSF from 3 individuals/patients. Statistical analysis was performed using one-way ANOVA with uncorrected Fisher's LSD test. Significance is shown with ***=P<0.0001, **=P<0.01 and *=P<0.05. FIG. 2 shows the increased pericyte proliferation upon exposure to MS serum. HBVP were seeded in xCELLigence E-plates and, after 24 hours, were exposed to (10%, v/v) either culture medium (control), healthy human serum (n=2 individuals), or MS patient serum (n=2 individuals) for 100 hours. Each line was determined by the average of duplicate wells. MS serum induced the proliferation rate of pericytes compared to culture medium or healthy serum. The proliferation rate was calculated by analyzing the slope between 40 and 80 hours, which was 0.022±0.002 cell index/hour (mean±SD for n=2 patients) for MS serum and 0.014±0.001 per hour (mean±SD for n=2 patients) for healthy serum. According to the data, it was revealed that pericytes, when exposed to MS serum (two-tailed unpaired T-test p-value: 0.032), proliferated significantly faster compared to healthy serum. FIGS. 3A-3H show the analysis of PLP EAE induced fibrotic areas in spinal cord and cerebellum sections. FIG. 3A: Body weight change and FIG. 3B: EAE score graphs for sham and PLP EAE induced mice for 40 days after the first immunization. Data is shown as mean±SEM for sham n=9 and PLP EAE n=9. Statistical significance was measured with Mixed-Effects model for Repeated Measures analysis with Bonferroni's multiple comparisons test. FIGS. 3C-3H: Total cell count is shown per mm2 area and the expression levels of Albumin, PDGFRβ, Collagen I, Collagen IV and Fibronectin are shown as Integrated density per μm2 area for early (15 days) and late (40 days) phase sham and EAE groups. Scatter plot diagrams are shown with mean±SD for n>3 animals and statistical analysis was performed by using two-way ANOVA with uncorrected Fisher's LSD test. All significance data is shown with ****=P<0.0001, ***=P<0.001, **=P<0.01 and *=P<0.05.

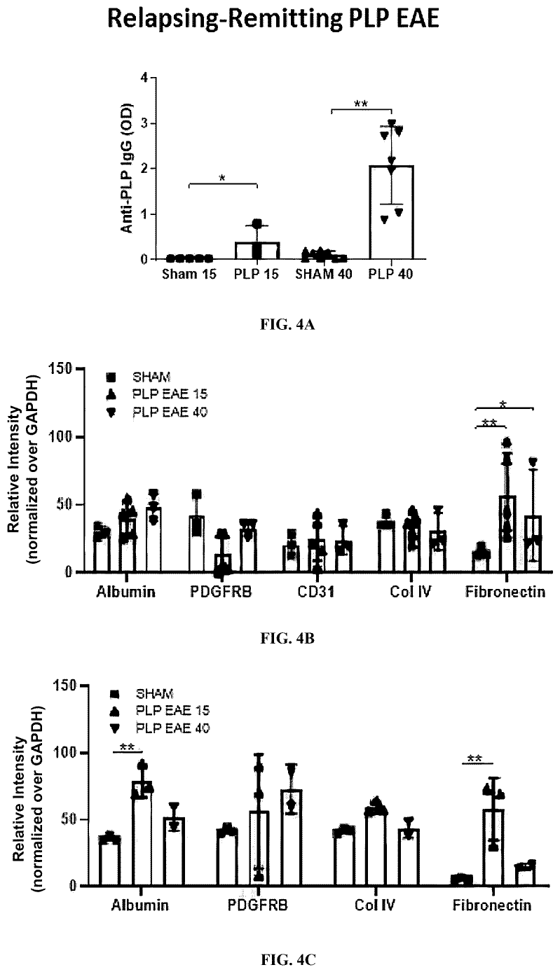

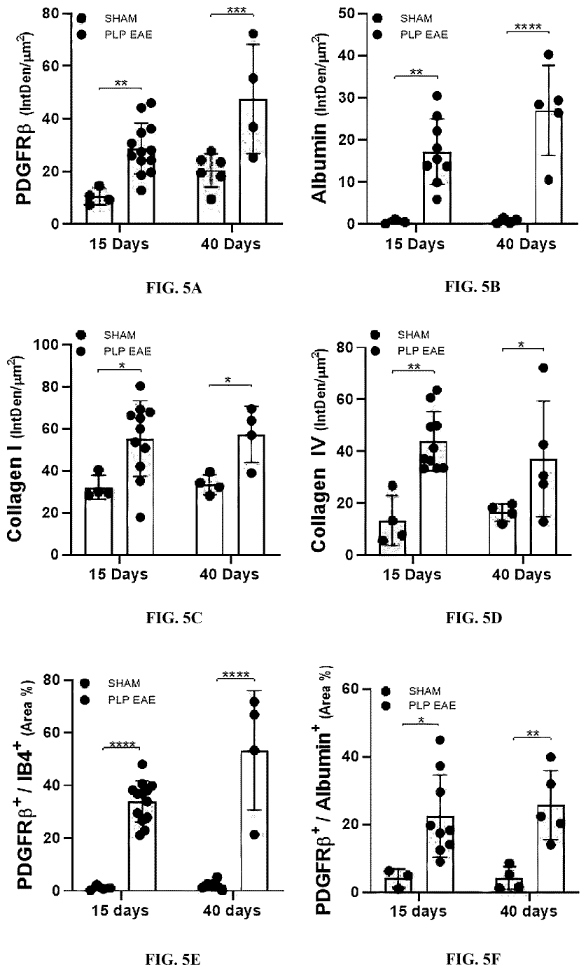

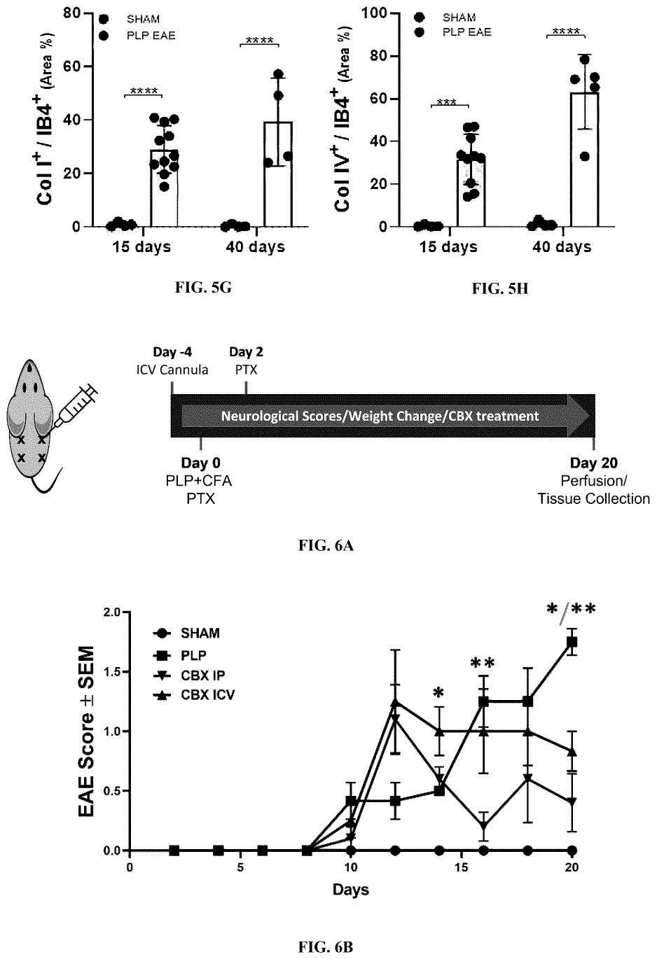

FIGS. 4A-4C show the increased fibrotic parameters in EAE. FIG. 4A: anti-PLP antibody titers in sera of sham and EAE induced mice. Scatter plot diagrams are shown with mean±SD for n≥4 animals. Statistical analysis was performed by using Kruskal-Wallis with uncorrected Dunn's test. FIG. 4B: Band intensity analysis of Western blot experiments; Albumin, PDGFRβ, CD31, Collagen IV, and Fibronectin normalized over internal GAPDH protein level in brain homogenates of sham and PLP EAE groups. Cross symbol in western blot lanes indicates mouse died before sacrificing. Scatter plot diagrams are shown with mean±SD for n=3 animals. Statistical analysis was performed by using two-way ANOVA with uncorrected Fisher's LSD test. FIG. 4C: band intensity analysis of western blot experiments; Albumin, PDGFRβ, Collagen IV, and Fibronectin normalized over internal GAPDH protein level in spinal cord homogenates of sham and PLP EAE groups. Scatter plot diagrams are shown with mean±SD for n≥2 animals. Statistical analysis was performed by using two-way ANOVA with uncorrected Fisher's LSD test. All significance data is shown with ****=P<0.0001, ***=P<0.001, **=P<0.01 and *=P<0.05. FIGS. 5A-5H show PDGFRβ+ perivascular cells tightly cover leaking vessels located in fibrotic areas in PLP EAE spinal cord sections. PDGFRØ is a known marker for human brain perivascular pericytes. FIG. 5A: PDGFRβ, FIG. 5B: Albumin, FIG. 5C: Collagen I and FIG. 5D: Collagen IV expression is shown as Integrated density per μm2 area for sham and PLP EAE groups. FIG. 5E: Overlapping areas of PDGFRβ+ over IB4+, FIG. 5F: PDGFRβ+ over Albumin+, FIG. 5G: Collagen I+ over IB4+ and FIG. 5H: Collagen IV+ over IB4+ is shown as area in percentages for sham and PLP EAE groups. Scatter plot diagrams are shown with mean±SD for n≥3 animals. Statistical analysis was performed by using two-way ANOVA with uncorrected Fisher's LSD test. All significance data is shown with ****=P<0.0001, ***=P<0.001, **=P<0.01 and *=P<0.05.

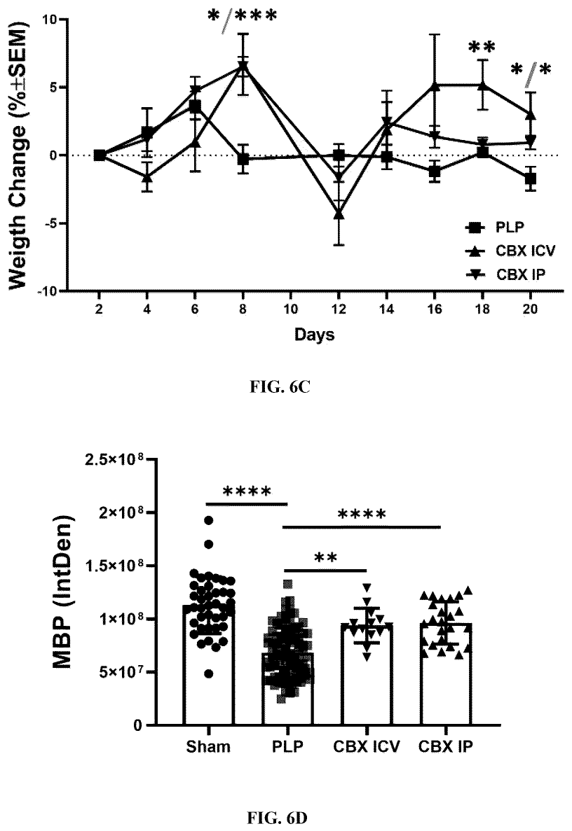

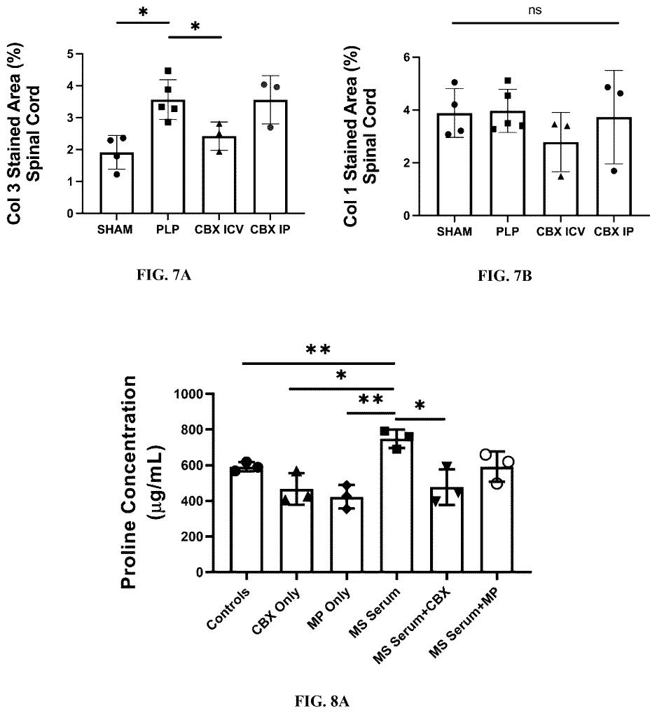

FIGS. 6A-6D show the reversal of disease progression by carbenoxolone treatment in PLP-EAE model. FIG. 6A: The injection sides and the timeline of the experiment are shown. FIG. 6B: The neurological scores deteriorated quickly at day 8 and entered remission from day 12 to the end of the experiment for CBX-treated groups but increased gradually and were maximum on the last day of the investigation for the PLP-EAE group. FIG. 6C: Weight gain followed a similar trend with the neurological scores, and the worst day (the maximum weight loss) was observed after the day with top neurological scores. FIG. 6D: The fluorescent intensity analysis of MBP, which was used for detecting demyelinated areas typical for Multiple sclerosis, revealed that it was significantly decreased in the PLP group compared to the other groups and both IP and ICV CBX treatment decreased demyelination (SHAM: 40 region, n=4; PLP: 85 region, n=5, CBX ICV: 24 region, n=3; CBX IP: 21 region, n=3). Data were presented as Mean±SEM for B and C, and Mean+SD for E. The significance of comparisons for PLP-EAE versus IP CBX was shown with black asterisks and ICV CBX versus PLP-EAE with red asterisks. *p<0.05, **<0.01. IntDen: Integrated Density.

FIGS. 7A-7B show reversal of the fibrosis upon treatment with intracerebroventricular carbenexolone treatment in EAE model. Collagen 3 and Collagen 1 stained spinal cord sections were analyzed for SHAM (control), PLP EAE mouse, CBX ICV and CBX IP treated groups. FIG. 7A: The image analysis results demonstrated that Collagen 3 stained area is significantly increased in the PLP group and successfully reduced in the CBX-ICV group (SHAM: 38 region, n=4, PLP: 76 region, n=5, CBX ICV: 27 region, n=3, CBX IP: 24 region, n=3). FIG. 7B: The Collagen 1 stained area was similar between groups (SHAM: 28 region, n=4, PLP: 30 region, n=5, CBX ICV: 24 region, n=3; CBX IP: 21 region, n=3). Data were presented as Mean=SD. NS: non-significant, *p<0.05.

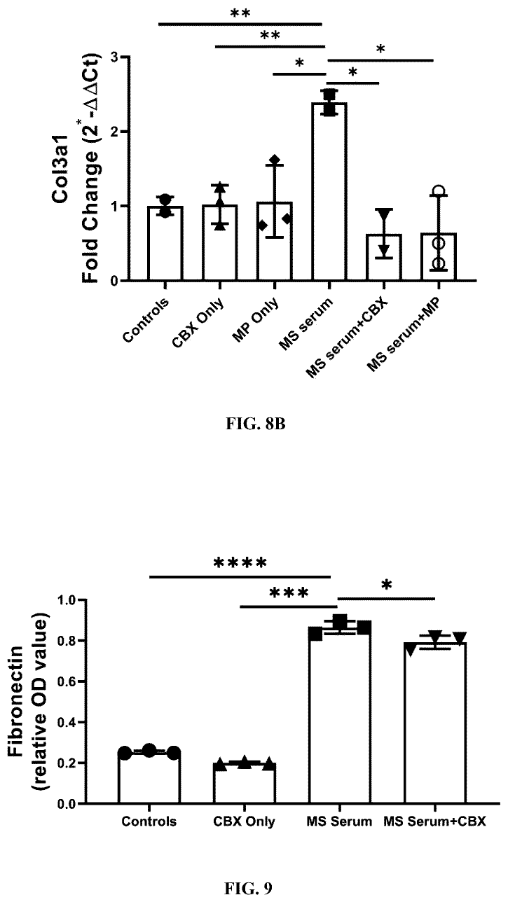

FIGS. 8A-8B show the collagen 3 expressions in pericyte culture treated with MS serum and reversal of fibrosis on pericytes upon exposure to carbenoxolone. FIG. 8A: Total proline concentration was measured with collagen assay. MS serum significantly increased proline concentration. And treatment with CBX but not MP decreased MS serum-induced overproduction. FIG. 8B: Col3a1 mRNA expression was studied with qPCR. Incubation with MS serum significantly increased the Collagen 3 gene expression. MS serum exposure increased the collagen 3 staining more than controls, and treatment with CBX but not MP decreased the MS Serum effect on Collagen 3 production. Data are presented as Mean±SD. *≤0.05, **<0.01.

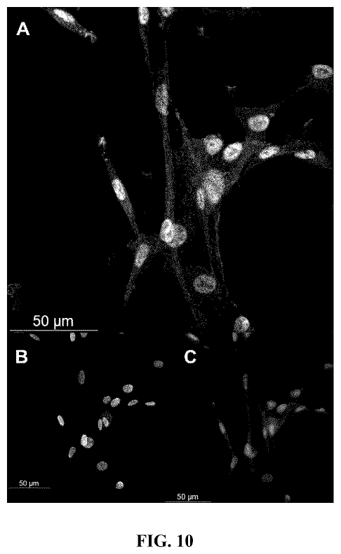

FIG. 9 shows the fibronectin expressions in pericyte culture treated with MS serum and decrease of fibronectin related fibrosis with treatment of carbenoxolone. Fibronectin levels were measured with ELISA. MS Serum significantly increased extracellular Fibronectin, and CBX treatment decreased this over-synthesis. MS serum exposure increased the Fibronectin staining more than controls, and treatment with CBX decreased the MS Serum effect on Fibronectin production. Data are presented as Mean±SD. *≤0.05, ***<0.001, ****<0.0001. OD: Optical Density.

FIG. 10 shows the presence of pannexin 1 (Panx1) channels on human brain vascular pericytes (HBVP). HBVPs were cultured in vitro and analyzed using confocal microscopy. The cells were stained with A) anti-Panx1 (to detect Panx1 channels), B) DAPI (to label nuclei), and C) anti-αSMA (a pericyte marker). Pannexin 1 channels, known targets of carbenoxolone, are demonstrated here for the first time on HBVPs.

Carbenoxolone can be administered either intrathecally for direct CNS access or systemically for broader availability, with intrathecal administration providing a targeted approach for CNS lesions and systemic administration supporting widespread bioavailability. In Mouse Experiments (20-day experiment) Carbenexolone was given to mice once every 2 days for 20 days starting from Day 0. The relapsing-remitting MS model was created by immunizing female BalbC mice with PLP antigen (FIG. 1A). For each animal, 100 μl of PLP139-151 dissolved in PBS at 1 mg/mL concentration (PLP-3812-PI from Peptides International, Louisville, KY) was emulsified in 100 μl of complete Freund's adjuvant (CFA) including 5.33 mg/mL of M. Tuberculosis H37. Under isoflurane anesthesia, a total of 200 μl was divided into four locations and administered subcutaneously. In addition, 250 ng of 5 μg/mL Pertussis toxin (PTX) (cat. no. P7208-50UG, Sigma-Aldrich) has injected IP each mouse 1 hour and two days after the vaccination. The experimental groups were Sham (n=6), PLP EAE (n=6), CBX IP (n=5), and CBX ICV (n=5). The sham group received the CFA adjuvant only without PLP. The CBX IP group received CBX at a dose of 20 mg/kg from 2.5 mg/ml intraperitoneally, and the CBX ICV group 25 μg/kg from 0.25 mg/ml intracerebroventricularly from the cannula every other day starting from day 0. All experimental animals' weight changes and EAE scores were monitored every two days for 20 days. In this experiment, the intraperitoneal route models systemic drug administration, whereas the intracerebroventricular (intrathecal) route models drug efficacy directly in the Cerebrospinal Fluid, crossing the blood brain barrier. In this context, it is possible that both routes may be used in future human trials, which will need to be confirmed by further human trials. For systemic administration in humans, intravenous, intramuscular or oral administration can be used, while for intrathecal administration, intrathecal injection from the lumbar region or Ommaya reservoir into the ventricle can be used.

To enhance Carbenoxolone's effectiveness, the formulation can include excipients that improve its ability to cross the BBB, ensuring adequate drug levels within the CNS. Carbenoxolone may also be combined with anti-inflammatory agents or other disease-modifying therapies (DMTs), allowing a dual-action approach that addresses both fibrosis and inflammation in MS, aiming to improve disease management. The excipients may be liposomes and/or nanoparticles. Liposomes are biocompatible nanoscale vesicles capable of crossing the blood-brain barrier (BBB) and delivering drugs to specific brain cells, including pericytes. Their structure, composed of lipid bilayers surrounding an aqueous core, allows for the encapsulation of both hydrophilic and hydrophobic drugs. To facilitate BBB crossing, liposomes can be modified with ligands or antibodies that exploit receptor-mediated transcytosis pathways, such as those involving transferrin or insulin receptors on endothelial cells. Once in the brain, their surface can be functionalized with ligands targeting receptors specifically expressed on pericytes, such as platelet-derived growth factor receptor-β (PDGFR-β). Additionally, stimuli-responsive liposomes, which release their drug cargo in response to environmental triggers like pH or enzymatic activity, enable localized and precise drug delivery. These properties make liposomes a promising platform for delivering drugs to treat neurological conditions while minimizing off-target effects.

Nanoparticles are highly versatile drug delivery systems that can be tailored to cross the BBB and reach pericytes with high specificity. Their structural diversity ranging from polymeric and metallic to lipid-based forms enables precise control over size, surface charge, and drug loading. Nanoparticles can be functionalized with ligands or antibodies that target BBB transport mechanisms, such as receptor-mediated or adsorptive-mediated transcytosis, ensuring efficient passage into the brain. For pericyte-specific targeting, nanoparticles can carry ligands that bind to unique surface markers like PDGFR-β. Additionally, their surface can be modified for stealth properties, such as PEGylation, to prolong circulation time and evade immune detection. Advanced nanoparticles can also incorporate diagnostic agents or be engineered to respond to external stimuli, such as magnetic fields or ultrasound, further enhancing their precision and versatility in drug delivery. These characteristics position nanoparticles as powerful tools for delivering therapeutic agents to pericytes and other specialized brain cells.

DMTs that can be used together are interferons, glatemir acetate, sphingosine-1-phosphate receptor modulators, teriflunomide, cladribine, fumarates, and monoclonal antibodies such as natalizumab, alemtuzumab, ocrelizumab, ofamtumumab. In conclusion, the experimental results demonstrated that Carbenoxolone (CBX) exhibits strong antifibrotic and neuroprotective properties in both in vitro and in vivo models of Multiple Sclerosis (MS). The in vitro experiments with human brain vascular pericytes, astrocytes, and endothelial cells exposed to MS patient serum showed a significant increase in extracellular matrix components such as Collagen IV and Fibronectin. CBX effectively reduced the overproduction of these fibrotic markers, indicating its potential to mitigate fibrosis within the blood-brain barrier (BBB) in MS. The in vivo results from the PLP-induced EAE model further validated CBX's efficacy, as CBX-treated groups exhibited reduced neurological deficits, less weight loss, and diminished collagen deposition in the spinal cord. CBX, particularly when administered intracerebroventricularly, significantly reduced perivascular fibrosis, preserved BBB integrity, and mitigated demyelination compared to untreated EAE mice. Moreover, CBX's ability to reduce Collagen 3 and Fibronectin production in both pericyte cultures and spinal cord sections underscores its potential as a novel therapeutic agent that directly targets the fibrotic processes contributing to MS progression. The combined data from these studies strongly support the therapeutic potential of CBX in managing MS by addressing both neurovascular and fibrotic mechanisms, which are often overlooked by conventional immune-modulating therapies Therefore, CBX could provide a comprehensive approach to MS treatment, focusing on long-term disease management by targeting both inflammation and fibrosis to protect CNS integrity.

Autoimmune inflammatory demyelinating diseases of central nervous system (CNS) that we aim to treat with carbenexolone include (this is not the complete list because not all demyelinating diseases are well studied, this drug, carbenexolone, as we have demonstrated can amilerioate fibrosis formation in all demyelinating diseases of CNS): Multiple Sclerosis, transverse myelitis, neuromyelitis optica spectrum disorder (NMOSD), acute disseminated encephalomyelitis (ADEM), and myelin oligodendrocyte glycoprotein antibody-associated disease (MOGAD).

Multiple sclerosis (MS) is a chronic autoimmune disorder of the central nervous system, characterized by inflammation, demyelination, blood-brain barrier disruption, gliosis, and neuronal loss. Common symptoms include visual disturbances, sensory deficits, muscle weakness, bladder and bowel dysfunction, and cognitive decline. MS is the most prevalent immune-mediated inflammatory demyelinating disease of the CNS, affecting approximately 400,000 people in the United States and 2.5 million worldwide. Onset typically occurs between the ages of 20 and 40. Treatment aims to induce remission, reduce relapse frequency, minimize MRI activity, and prevent long-term disability. Currently, disease-modifying therapies, focusing on suppressing the autoimmune activity, serve as the primary therapeutic approach to alter disease progression and improve long-term outcomes [5].

Neuromyelitis optica spectrum disorder (NMOSD) is a rare inflammatory demyelinating disease of the central nervous system, most commonly presenting with visual loss, cranial nerve deficits, muscle weakness, and sensory disturbances. Unlike multiple sclerosis, NMOSD is strongly associated with aquaporin-4 immunoglobulin G (AQP4-IgG) antibodies, making serologic testing essential for diagnosis. Core clinical features include optic neuritis, longitudinally extensive transverse myelitis, brainstem syndrome, and area postrema syndrome. The global prevalence ranges from 0.3 to 4.4 per 100,000, with a marked female predominance (approximately 80%) and typical onset between ages 30 and 40. The proportion of NMOSD among demyelinating disorders varies widely—1-2% in the United States and Italy, but up to 13.7% in India and over 30% in Thailand. Long-term management centers on immunosuppressive therapy, such as azathioprine or rituximab, to reduce relapse risk and prevent irreversible neurological damage [6].

Myelin oligodendrocyte glycoprotein antibody-associated disease (MOGAD) is a recently recognized inflammatory demyelinating disorder of the central nervous system. Clinically distinct from both multiple sclerosis (MS) and AQP4-IgG-positive neuromyelitis optica spectrum disorder (NMOSD), MOGAD presents with a wide range of symptoms, including optic neuritis, transverse myelitis, acute disseminated encephalomyelitis (ADEM), and cortical encephalitis. Around half of patients experience relapsing disease. Diagnosis relies on detecting MOG-IgG antibodies-typically in serum- and identifying characteristic MRI findings. However, false positives can occur, especially with low antibody titers or indiscriminate testing. Unlike MS and NMOSD, MOGAD shows no significant gender bias and can affect all age groups, though it is more common in children. Its incidence is low, roughly 1.6 per million annually, and varies by region and ethnicity. Treatment of acute attacks generally includes high-dose corticosteroids, with plasma exchange for refractory cases. Long-term immunosuppression is considered for relapsing disease or patients with severe outcomes, although evidence-based guidelines are still evolving [7].

Transverse myelitis (TM) is a rare disease affecting a specific segment of the spinal cord, typically presenting with sudden onset of weakness, sensory disturbances, and bowel or bladder dysfunction. While it often occurs as an isolated event, TM can also arise as part of broader neuroinflammatory diseases or as a post-infectious complication. It can affect any region of the spinal cord, with the thoracic segment being the most commonly involved. The disease course ranges from temporary symptoms lasting 3 to 6 months to potentially permanent disability. TM affects both sexes equally and shows a bimodal age distribution, with peaks between 10-19 and 30-39 years. Its incidence is estimated at 1 to 8 new cases per million people annually. Acute attacks are typically managed with high-dose corticosteroids or plasma exchange, while recurrent or severe cases may benefit from long-term immunosuppressive therapies such as cyclophosphamide, mycophenolate mofetil, or rituximab[8].

Acute disseminated encephalomyelitis (ADEM) is a rare, rapidly progressing autoimmune disorder characterized by widespread demyelination of the brain and spinal cord, typically triggered by a prior infection or, less commonly, vaccination. The condition is most often seen in children, with an annual incidence of 0.07 to 0.9 per 100,000. It commonly affects children between the ages of 5 and 8, with most cases occurring under age 10, though it can also be seen in adolescents and adults ranging from 18 to 82 years. ADEM affects all ethnic groups globally, though lighter skin pigmentation and certain genetic and environmental factors may influence susceptibility. In 50% to 85% of cases, ADEM is preceded by a viral or bacterial infection or, less frequently, immunization. Treatment primarily involves high-dose corticosteroids, while intravenous immunoglobulin (IVIG) and plasma exchange are considered in severe or refractory cases[9].

### REFERENCES

[1] McGinley, M. P., Goldschmidt, C. H., & Rae-Grant, A. D. (2021). Diagnosis and treatment of multiple sclerosis: A review. JAMA, 325 (8), 765-779.
[2] Ruiz, F. C., Vigne, S., & Pot, C. (2019). Resolution of inflammation during multiple sclerosis. Seminars in Immunopathology, 41 (6), 711-726.
[3] Buscarinu, M. C., Reniè, R., Morena, E., Romano, C., Bellucci, G., Marrone, A., et al. (2022). Late-onset MS: disease course and safety-efficacy of DMTS. Front. Neurol. 13:9331. doi: 10.3389/fneur.2022.829331
[4] Özkan, E., çetin-Taş, Y., Şekerdağ, E., & Gökyüzü, A. B. (2021a). Blood-brain barrier leakage and perivascular collagen accumulation in multiple sclerosis. Metabolic Brain Disease, 36 (6), 2553-2566.
[5] Tafti D, Ehsan M, Xixis K L. Multiple Sclerosis. [Updated 2024 Mar. 20]. In: StatPearls [Internet]. Treasure Island (FL): StatPearls Publishing; 2025 January. Available from: www.ncbi.nlm.nih.gov/books/NBK499849/
[6] Shumway C L, Patel B C, Tripathy K, et al. Neuromyelitis Optica Spectrum Disorder (NMOSD) [Updated 2024 Jan. 8]. In: StatPearls [Internet]. Treasure Island (FL): StatPearls Publishing; 2025 January. Available from: www.ncbi.nlm.nih.gov/books/NBK572108/
[7] Sechi E, Cacciaguerra L, Chen J J, Mariotto S, Fadda G, Dinoto A, Lopez-Chiriboga A S, Pittock S J, Flanagan E P. Myelin Oligodendrocyte Glycoprotein Antibody-Associated Disease (MOGAD): A Review of Clinical and MRI Features, Diagnosis, and Management. Front Neurol. 2022 Jun. 17; 13:885218. doi: 10.3389/fneur.2022.885218. PMID: 35785363; PMCID: PMC9247462.
[8] Simone C G, Emmady P D. Transverse Myelitis. [Updated 2022 Nov. 15]. In: StatPearls [Internet]. Treasure Island (FL): StatPearls Publishing; 2025 January. Available from: www.ncbi.nlm.nih.gov/books/NBK559302/
[9] Anilkumar A C, Foris L A, Tadi P. Acute Disseminated Encephalomyelitis. [Updated 2024 Jan. 26]. In: StatPearls [Internet]. Treasure Island (FL): StatPearls Publishing; 2025 January. Available from: www.ncbi.nlm.nih.gov/books/NBK430934/

What is claimed is:

1. A method for treating a demyelinating neurological disorder, comprising administering Carbenoxolone to a subject, wherein the Carbenoxolone reduces a perivascular fibrosis within a central nervous system, thereby preserving a blood-brain barrier integrity and inhibiting a lesion formation, Formula I of the Carbenoxolone is shown below:

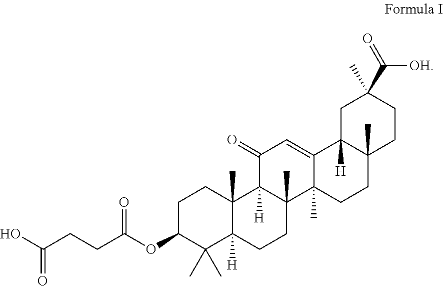

2. The method according to claim 1, wherein the Carbenoxolone is administered intrathecally or systemically, wherein an intrathecal administration allows a direct access to the central nervous system.

3. The method according to claim 1, wherein the Carbenoxolone is combined with liposomes and/or nanoparticles as excipients to enhance bioavailability.

4. The method according to claim 1, wherein the Carbenoxolone is combined with anti-inflammatory agents or other disease-modifying therapies (DMTs) to enhance an efficacy of the Carbenoxolone by addressing both fibrotic and inflammatory processes in Multiple Sclerosis (MS), wherein the DMTs allowed to be used together comprise interferons, glatemir acetate, sphingosine-1-phosphate receptor modulators, teriflunomide, cladribine, fumarates, and monoclonal antibodies, wherein the monoclonal antibodies comprise natalizumab, alemtuzumab, ocrelizumab, and ofamtumumab.

5. The method according to claim 1, wherein the demyelinating neurological disorder is MS.

6. The method according to claim 2, wherein the demyelinating neurological disorder is MS.

7. The method according to claim 3, wherein the demyelinating neurological disorder is MS.

8. The method according to claim 4, wherein the demyelinating neurological disorder is the MS.
